(19) United States
(12) Patent Application Publication
(10) Pub. No.: US 2013/0267939 A1
(43) Pub. Date: Oct. 10, 2013

(54) Methods and Apparatus for Ablating Lung Nodules with Vapor

(71) Applicants: Robert Lawrence Barry, Kirkland, WA (US); Brian Thomas Cran, Seattle, WA (US); Dean T. Corcoran, Bothell, WA (US); Daniel Lawrence Reddy, Seattle, WA (US); Erik Henne, Seattle, WA (US)

(72) Inventors: Robert Lawrence Barry, Kirkland, WA (US); Brian Thomas Cran, Seattle, WA (US); Dean T. Corcoran, Bothell, WA (US); Daniel Lawrence Reddy, Seattle, WA (US); Erik Henne, Seattle, WA (US)

(21) Appl. No.: 13/860,420

(22) Filed: Apr. 10, 2013

Related U.S. Application Data
(60) Provisional application No. 61/622,392, filed on Apr. 10, 2012.

(51) Int. Cl. A61B 18/04

(52) U.S. Cl. CPC A61B 18/04
USPC 606/27

(57) ABSTRACT

Devices and systems are described for lung nodules with heated condensable vapor. The device can have an elongated shaft coupled to a vapor generator. High temperature vapor can be directed through the shaft into the lung to treat the lung nodule. In some embodiments, the device comprises a needle configured to deliver the vapor to the lung nodules. An expandable member, such as a balloon, can be provided on a distal portion of the shaft to prevent proximal flow of the high temperature vapor upon discharge from the device. Methods of use are also provided.

### CROSS-REFERENCE TO RELATED APPLICATIONS

This application claims priority to U.S. Provisional No. 61/622,392, filed Apr. 10, 2012, titled “Methods and Apparatus for Ablating Lung Nodules with Vapor”, which is incorporated herein by reference in its entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD OF THE INVENTION

The present disclosure generally relates to treatment of lung disease such as cancer or tuberculosis. More specifically, the present disclosure relates to treatment of a nodule or nodules within the lung with a heated condensable vapor.

### BACKGROUND

A typical surgical treatment of lung cancer is to resect malignant nodule and surrounding tissue. In addition to the nodule, a margin is also resected in an attempt to ensure that all cancerous cells have been eliminated. Often an entire lobectomy is performed. These surgical procedures are expensive, tedious, and not recommended for many lung cancer patients due to the high risk of complication of surgery.

A surgical treatment for tuberculosis is to resect nodules containing active tubercle bacilli. In addition to the nodule, a margin is also resected in an attempt to ensure that the active tubercle bacilli is eliminated. Often an entire lobectomy is performed. These surgical procedures are expensive, tedious, and not recommended for many lung cancer patients due to the high risk of complication of surgery.

Heating therapies are increasingly used in various medical disciplines including cardiology, dermatology, orthopedics, oncology as well as a number of other medical specialties. In general, the manifold clinical effects of superphysiological tissue temperatures results from underlying molecular and cellular responses, including expression of heat-shock proteins, cell death, protein denaturation, tissue coagulation and ablation. Associated with these heat-induced cellular alternations and responses are dramatic changes in tissue structure, function and properties that can be exploited for a desired therapeutic outcome such as tissue injury, shrinkage, modification, destruction and/or removal.

Heating techniques in the lung pose several technical challenges because lung tissue is more aerated than most tissues and also due to its vascularization. Accordingly, new heating methods, devices and systems for rapid, controllable, effective and efficient heating of lung tissue are needed.

### SUMMARY OF THE DISCLOSURE

A method of applying energy to tissue of a patient's lung to treat lung disease is provided, the method comprising identifying at least one lung region having a diseased nodule to be treated, inserting a vapor delivery needle into the lung region, and delivering vapor through the vapor delivery needle into the lung region to ablate the diseased nodule.

In some embodiments, the lung region is a lung segment or sub-segment. In another embodiment, the diseased nodule can be a cancer nodule.

In some embodiments, the method further comprises heating the vapor to at least 100° C. before delivering the vapor. The method can further comprise ablating tissue surrounding the diseased nodule with the vapor.

In one embodiment, the method further comprises determining a predetermined dose of vapor to be delivered to the lung region. In some embodiments, the determining step comprises determining the dose of vapor to be delivered based on the mass of the nodule. In another embodiment, the determining step comprises determining the dose of vapor to be delivered based on the diameter of the nodule.

In some embodiments, the method further comprises terminating delivery of vapor to the lung region when a temperature of the diseased nodule reaches a predetermined threshold.

In one embodiment, energy transferred to the tissue by the vapor causes coagulative necrosis of the nodule.

In some embodiments, the step of delivering the vapor comprises delivering the vapor at a flow rate of between about 20 calories/second to about 200 calories/second. In another embodiment, delivering the vapor comprises delivering the vapor for a duration of between about 2 seconds to about 5 minutes. In yet another embodiment, delivering the vapor comprises delivering the vapor for a duration of between about 4 seconds to about 10 seconds.

In another embodiment, the method comprises generating the vapor in a generator disposed outside of the patient.

In one embodiment, the dose is between about 5 cal/g and about 20 cal/g. In another embodiment, the dose is between about 5 cal/g and about 10 cal/g. In yet another embodiment, the dose is between about 20 cal/g and about 40 cal/g.

In some embodiments, the inserting step comprises forming a channel into the lung region with the vapor delivery needle. In another embodiment, the forming a channel step comprises forming a channel by puncturing through a pleural surface.

In some embodiments, the inserting step comprises puncturing the lung with the vapor delivery needle.

In some embodiments, the method further comprises adjusting a vapor flow parameter during the delivering step to increase precision of vapor delivery. In one embodiment, the adjusting step comprises adjusting a vapor quality. In another embodiment, the adjusting step comprises adjusting a vapor momentum. In additional embodiments, the adjusting step comprises pulsing a vapor quality, a vapor flow rate, or a vapor momentum.

In some embodiments, the inserting step comprises inserting the vapor delivery needle directly into the diseased nodule. In yet another embodiment, the inserting step comprises positioning the needle near the diseased nodule, and wherein the delivering step comprises delivering vapor through a parenchyma of the lung to ablate the diseased nodule.

In another embodiment, the delivering vapor step comprises delivering vapor through the vapor delivery needle directly into the diseased nodule to ablate the diseased nodule.

In another embodiment, the method comprises, prior to the inserting step, forming a channel in a parenchyma of the lung. In some embodiments, the inserting step comprises positioning the needle near the diseased nodule, and wherein the delivering step comprises delivering vapor through the channel in the parenchyma to ablate the diseased nodule.

Another method of treating disease of the lung is provided, the method comprising imaging at least one nodule to be treated of the lung tissue, determining a mass or a diameter of the nodule to be treated based on the imaging, determining a safe and efficacious dosage of vapor for treating the nodule based on the mass or diameter of the nodule, inserting a vapor delivery system into the lung proximate to the nodule to be treated, and delivering the safe and efficacious dosage of vapor from the vapor delivery system to the nodule to be treated to ablate the nodule.

In some embodiments, the inserting step further comprises inserting a vapor delivery needle of the vapor delivery system into the lung proximate or within to the nodule to be treated.

A system for treating disease of the lung is provided, the system comprising a vapor generator adapted to generate a heated water vapor, a delivery catheter coupled to the vapor generator, a vapor delivery needle disposed on a distal end of the delivery catheter, the vapor delivery needle configured to be inserted into a lung region, and an electronic controller configured to determine a dosage of vapor to be delivered to a lung nodule based on a mass or diameter of the nodule, the controller also configured to determine a duration for delivering the vapor based on the dosage and an energy flow rate of the vapor generator.

### BRIEF DESCRIPTION OF THE DRAWINGS

FIG. 1 is one embodiment of a vapor delivery system.

FIG. 2 is another view of the vapor delivery system of FIG. 1.

FIG. 3 is another embodiment of a vapor delivery system.

FIG. 4 is one view of a user interface for a vapor delivery system.

FIG. 5 illustrates a vapor delivery system delivering vapor to a patient's lung.

### DETAILED DESCRIPTION

An alternative is to thermally ablate a nodule and diseased tissue using thermal vapor. This document summarizes access methods, delivery methods, dosing, and vapor flow parameters of ablating diseased tissue with thermal vapor.

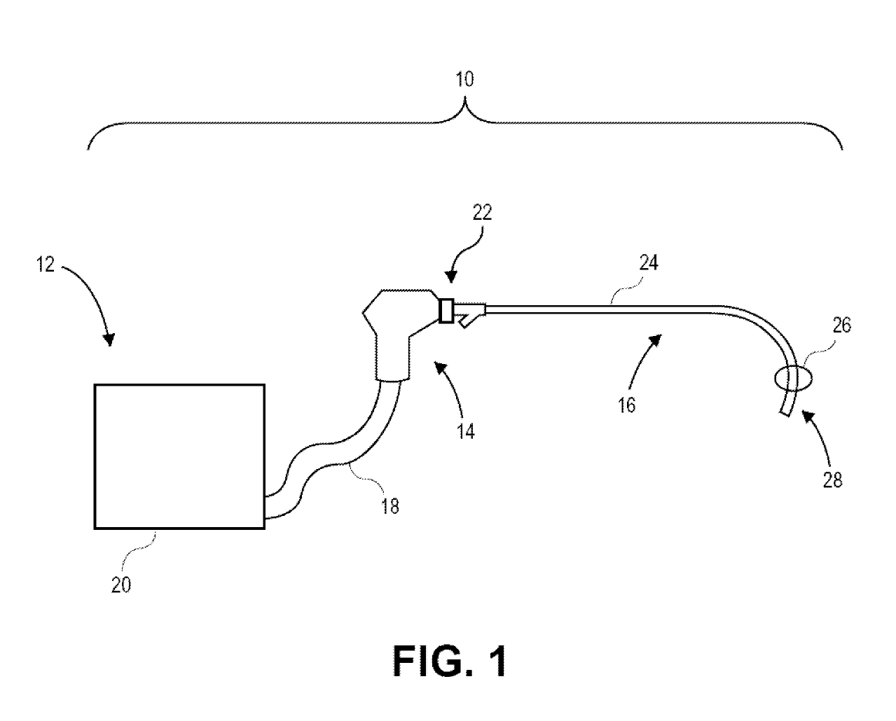

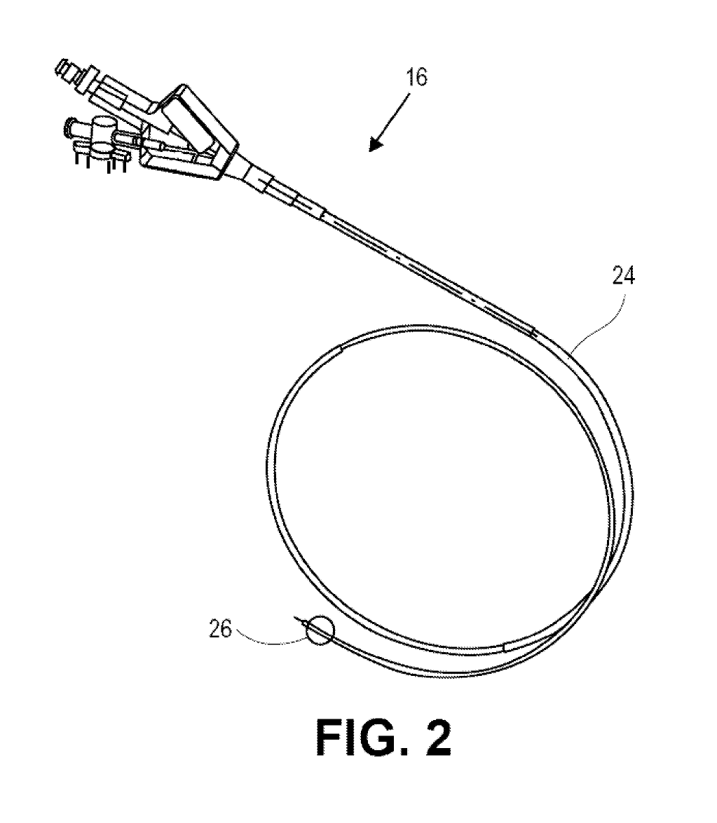

FIGS. 1-2 show one embodiment of a system 10 and system components for generating and delivering vapor to lung tissue to be treated. The system 10 generally comprises a vapor generator 12, hand-piece 14, and delivery catheter 16.

The vapor generator 12 can be attached to hand-piece 14 by tube 18. The generator can comprise a pressure vessel 20 containing liquid water (or other biocompatible liquid, such as saline) and a heating element (not shown) configured to heat the water or other biocompatible liquid to generate condensable vapor. In some embodiments, the vapor generator can further comprise sensors, such as temperature sensors, pressure sensors, and/or flow rate sensors, and valves to control the flow of vapor. Hand piece 14 can be coupled to the proximal end 22 of catheter 16.

The catheter is generally used to deliver the heated condensable vapor (steam) to a targeted segment or sub-segment of the subject's lung containing the diseased lung nodule. A diseased lung nodule can comprise, for example, a cancerous lung nodule or malignant tumor, or alternatively, a benign mass in the lung. The catheter 16 generally comprises flexible shaft 24 and occlusion balloon 26 located at or slightly proximal to the distal end 28 of the catheter.

In some embodiments, the catheter can be introduced to a lung segment via the airway using a bronchoscope. Once a target airway is reached that leads to a nodule, vapor can be delivered to ablate the nodule and the surrounding tissue using the airway as a delivery channel or the open parenchymal space. This can be done with and without the occlusion balloon shown in FIGS. 1-2.

One limitation of the bronchoscope method is the diameter of the scope, since a bronchoscope cannot access airways smaller in diameter than the outer diameter of the bronchoscope. One method of delivering a more precise treatment is to access a more distal (and smaller) airway. To do this, a pulmonary navigation system could be used. There are a number of ways to implement this.

In one embodiment, system 10 of FIGS. 1-2 can further include a navigation catheter (not shown) with a working channel. First, the navigation catheter can be navigated to the target airway using its navigation system. After the target airway is reached, the vapor catheter 16 can be pushed through the working channel to the target airway and to deliver vapor to ablate the tissue.

Another method is to incorporate the navigation system described above into the vapor catheter 16 of system 10. In this embodiment, the vapor catheter 16 is navigatable to the target tissue, without the need for a separate navigation catheter or bronchoscope to access the target site of the lungs. In this embodiment, the catheter is navigated to the target and then vapor is delivered to ablate the lung tissue.

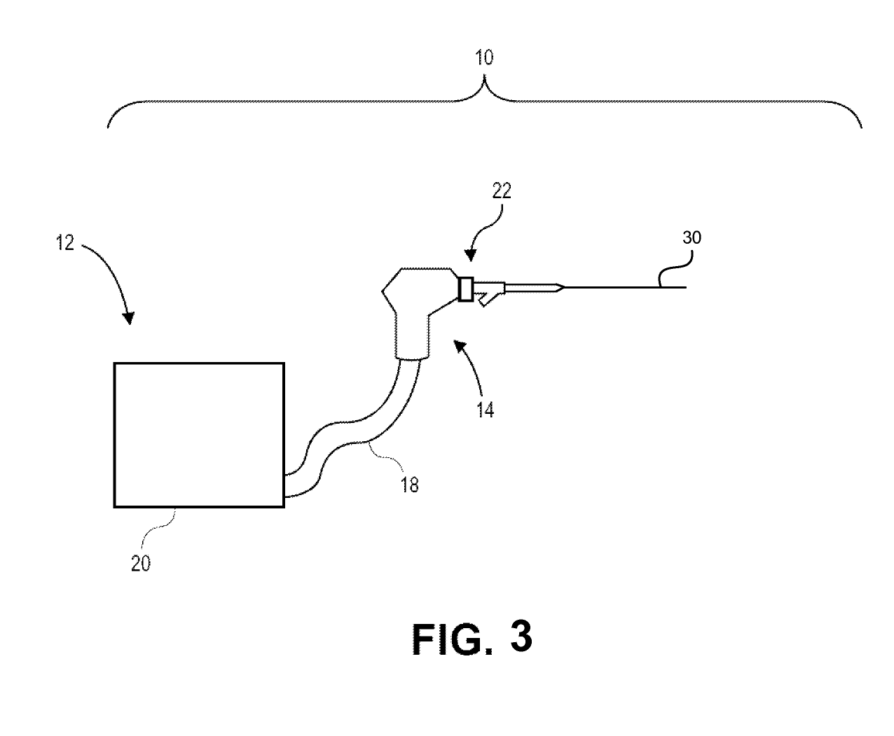

In yet another embodiment, rather than accessing target tissue through the airway tree as described above, it is also possible to access the target directly through the thoracic wall. Referring to FIG. 3, the system 10 can include a vapor delivery needle 30 in place of the vapor catheter 16 of the system of FIG. 1. In use, a channel can be created into the lung using needle 30. The location of the channel can be confirmed using navigation or imaging systems. Vapor from generator 12 can be delivered to the target tissue using the same vapor needle 30. In some embodiments, vapor can be delivered using a second needle/rigid catheter after the initial puncture is created. Vapor can be delivered by first introducing a working channel (either the original puncture needle or a new channel). A dedicated rigid or flexible catheter with or without a navigation system can then be introduced through this working channel to the targeted tissue. Target tissue can also be accessed by puncturing through an airway or pleural surface to reach parenchyma directly. Once in the parenchyma, vapor can be delivered through the needle to treat targeted tissue. To enhance precision to the targeted tissue, artificial “airway(s)” can be created using the needle or something equivalent.

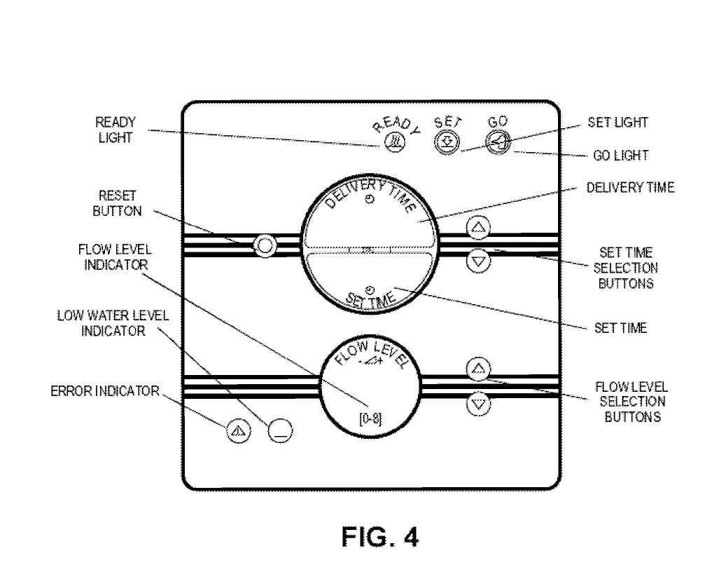

The vapor generator 12 of system 10 can be an electronically controlled pressure vessel configured to generate and deliver precise amounts of condensable vapor via the catheter to lung tissue. In some embodiments, the operator may select the flow level and the duration of the vapor treatment (the determination of which is described below) using a user interface on the front panel. An exemplary user interface is shown in FIG. 4. The combination of flow level and delivery time delivers a specific amount of vapor therapy to the patient. While delivery of vapor to the patient is preferably manually triggered by the operator using the handpiece, an electronic controller inside the generator can continuously monitor temperatures, pressures, water level, to ensure safety of the software. The user interface may further include flow level indicators, delivery time, errors in the system indicators, low water/fluid indicators, and a “ready” indicator when the system is ready to deliver therapy to a patient.

The vapor is generally heated to between about 100° C. to about 200° C. in the vapor generator. Vapor generated in a remote boiler will typically have a lower temperature upon delivery, but the vapor will still have a temperature at or above at least 100° C.

Referring again to FIGS. 1-2, the vapor catheter is preferably non-reusable and supplied sterile. The catheter can comprise components for occluding the target airway and delivering a dose of vapor from the vapor generator to the targeted lung segment or sub-segment. The catheter shaft can be adapted to allow delivery of the catheter through a bronchoscope, and the catheter comprises a balloon near the distal end of the catheter shaft to allow proper sealing of the targeted bronchi.

A general method of delivering vapor to the lung includes advancing the catheter into the region of the lung targeted for treatment, such as a segment or sub-segment of the lung. The balloon 26 at or near the distal end of the catheter tip can be inflated to seal the airway. The vapor can then delivered from the distal end of the catheter to the targeted tissue. After treatment, the balloon can then deflated to allow for withdrawal of the catheter.

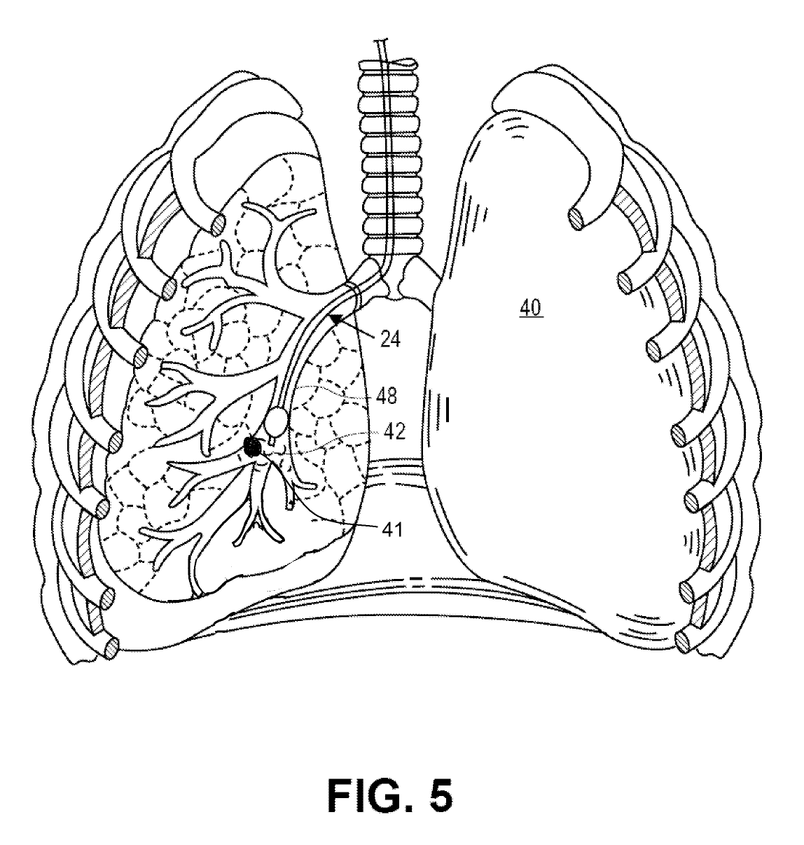

FIG. 5 illustrates one method of treating a patient's lung 40 embodying features of the invention that includes delivering a condensable vapor 42 to the airways 48 of lung tissue having a lung nodule 41, so as to create necrosis of the tissue of the nodule, the tissue of terminal bronchioles, and parenchymal tissue. The lung 40 and nodule 41 of the patient can be accessed with any of the systems described above in FIGS. 1-4, and as described below.

In one embodiment, the catheter based system of FIGS. 1-2 can be used to access the lung tissue, either with or without the aid of a bronchoscope. The distal tip of the catheter can be placed in proximity to the target nodule, or can be placed proximate from the nodule. In some embodiments, the distal tip of the catheter can be placed 0-20 cm from the nodule. In one embodiment, the tip of the catheter can be placed anywhere within the associated segment or lobe, from being physically inserted into the nodule, to treating at the base of the segmental airway, to treating from the pleural surface. Physical distance can depend on the size of the lung. Vapor generated by generator 12 can be delivered through the catheter to ablate the tissue of the lung nodule.

In another embodiment, referring to the needle based system of FIG. 3, the needle can be used to penetrate the lung tissue adjacent or close to the target nodule to be treated. For example, a lung nodule of a lung can be treated by identifying at least one lung region having a diseased nodule to be treated (e.g., such as by imaging with a chest X-ray or CT scan, or alternatively, via a Bronchoscopy). Next, a method can include inserting a vapor delivery needle into the lung region, then delivering vapor through the vapor delivery needle into the lung region to ablate the diseased nodule. The amount of vapor delivered to treat the nodule can be predetermined based on the mass or diameter of the nodule, for example.

Vapor can be delivered using the airways that naturally exist in the lung to ablate the diseased tissue. Airways are already very effective at transporting gases. In the case where an airway does not lead to the nodule, a pathway or channel can be created using a needle or other channeling device through the airway wall and into the parenchyma to allow the vapor catheter to traverse into. Depending on the location and size of the nodule, multiple treatments in multiple airways or through multiple channels may be required to ablate a necessary amount of the nodule and margin.

Vapor energy is typically measured in calories. The proper dose of vapor energy to be delivered can be determined using a number of methods. Dosing can be made more precise by adding further factors to the algorithms such as distance of the nodule from pleura, availability of airways, disease state, airway diameters, tissue density, and blood perfusion.

In some embodiments, the dosing of vapor to treat a nodule can be calculated with a mass-based system. First, the volume of a target nodule can be determined with a CT or other imaging system. Next, the mass of the nodule can be calculated using the volume of the nodule from the imaging step and the density of the target tissue. A mass-based dose of energy can then be calculated using the following basic algorithm:

Calories to deliver=Dose (calories/gram)*Mass of target tissue (grams)

In another embodiment, a diameter-based system can be used to determine the dosage of vapor to deliver to the nodule. Again, using a CT or other imaging system, the diameter of a target nodule can be determined. A diameter-based dose of energy could then be calculated using the following basic algorithm:

Calories to deliver=Dose (calories/millimeter̂3)*diameter of nodule (millimeters)̂3

The ultimate goal of tissue ablation is to raise the temperature of the tissue past a certain threshold that will cause the desired effect of necrosis. Therefore, in another embodiment, an appropriate dose can be determined by delivering energy while simultaneously measuring temperature of the tissue or nodule. Energy delivery can be terminated once the predetermined threshold has been reached. In this case, a dose would be delivered using the following rule: Deliver energy using predetermined method and flow parameter until temperature of tissue measured reaches a predetermined threshold. In some embodiments, the preferred amount of calories to deliver to the nodule depends on the dose and the size of the nodule. Doses can range from 5 to 100 calories/gram (when dosing on mass). For example, a relatively small nodule can be 5 grams, and a large nodule may be around 100 grams. A dose at the low end can be around 25 calories, and a dose at the high end can be around 1000 calories. In some embodiments, nodules may need to be treated more than once per session. Alternatively, energy may need to be delivered in increments. In some embodiments, a margin around the nodule may need to be treated, which would increase the number of calories required.

Additionally, to increase the precision of dosing, the energy rate or flow parameter could be modified as the measured temperature changes before ultimately finishing treatment. These parameters can be modified either manually by the user, or automatically by the controller in the generator 12 based on system feedback. The temperature can be measured using a number of methods including, but not limited to, placing thermocouples on the catheter or in the lung, or placing temperature measurement seeds on the catheter or in the lung. In the seed embodiment, one or multiple seeds could be placed in or near the nodule. The seeds can be configured to report a temperature wirelessly to the controller of system 10. The seeds can be left in the tissue permanently or removed after the procedure.

In another embodiment, passive temperature measurement can be used that reports only when a certain temperature threshold has been crossed. This could be accomplished using a thermocromatic material, a temperature sensitive seed, a material that changes shape at a certain temperature, or other methods of demonstrating that a temperature threshold has been crossed.

A number of vapor flow parameters can be adjusted during delivery of vapor to the lung tissue to increase precision of vapor delivery. These parameters can be tailored for each patient. Additionally, the parameters can be modified during the treatment to increase precision of the therapy. These parameters can also be included in the dosing algorithms described above. The primary result that is changed by modifying these flow parameters is the “focus” of the tissue ablation i.e. increasing or decreasing the volume of tissue ablated. Other results that can be changed with flow parameters include: temperature change, temperature change rate, duration of temperature achievement, and thermal profile.

One example of a parameter that can be adjusted is the vapor quality of the system, which is the ratio of gas to liquid in the vapor. A higher vapor quality indicates a higher percentage of gas. In some embodiments, the vapor quality can range from 10%-98%. In additional embodiments, the vapor quality can range from 80-90%. Vapor quality can be modified by changing the boiler pressure and modifying the delivery catheter channel geometry. A higher vapor quality is more energy dense and less mass dense.

Another parameter is the energy flow rate. The rate of thermal energy leaving a catheter can be measured. This energy flow rate can be increased by increasing the back pressure, increasing the vapor quality, or modifying the delivery catheter channel (i.e., larger diameter, shorter distance). A higher energy flow rate will ablate tissue quicker. When flow rate is measure in terms of back pressure, the pressure can range from 10-200 psi. The flow and/or momentum depends on the orifice size and vapor quality.

Another parameter that can be adjusted is the momentum of the vapor, which is the speed of the vapor multiplied by the mass of the vapor. The vapor speed can be modified similarly to energy flow rate. The mass of the vapor can be modified by adjusting the vapor quality. A higher vapor momentum will affect the distance the vapor travels, where the energy is focused, and how even the energy is distributed within the tissue. Momentum can be described in terms of energy per second. In some embodiments, this momentum parameter can be in the range of 0.01-1 gram/second.

Another parameter that can be adjusted is the rate of the vapor energy delivery. The vapor energy rate can be modified by changing flow rate or vapor quality. A higher vapor energy rate will ablate tissue quicker, will affect where the energy is focused, and how even the energy is distributed within the tissue. Vapor energy rate can be described in terms of energy per second. In some embodiments, this energy rate momentum parameter can be in the range of 10-200 calories/second.

In another embodiment, instead of delivering a constant flow of vapor, vapor can be modified by pulsing the vapor delivery, vapor quality, flow rate, or vapor momentum. The pulse can follow a step function, sine function, or other pulse function. The pulse can be brought to zero between peaks or simply reduced. The pulse can be adjusted by modifying the amplitude, period, and slope of the function.

Adjustment of the various parameters described above can result in a duration of treatment between approximately 2 seconds to 5 minutes of vapor delivery to treat a diseased nodule.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method of applying energy to tissue of a patient's lung to treat lung disease, the method comprising:
identifying at least one lung region having a diseased nodule to be treated;
inserting a vapor delivery needle into the lung region; and
delivering vapor through the vapor delivery needle into the lung region to ablate the diseased nodule.

2. The method of claim 1 wherein the lung region is a lung segment or sub-segment.

3. The method of claim 1 further comprising heating the vapor to at least 100° C. before delivering the vapor.

4. The method of claim 1 further comprising ablating tissue surrounding the diseased nodule with the vapor.

5. The method of claim 1 further comprising determining a predetermined dose of vapor to be delivered to the lung region.

6. The method of claim 5 wherein the determining step comprises determining the dose of vapor to be delivered based on the mass of the nodule.

7. The method of claim 5 wherein the determining step comprises determining the dose of vapor to be delivered based on the diameter of the nodule.

8. The method of claim 5 wherein further comprising terminating delivery of vapor to the lung region when a temperature of the diseased nodule reaches a predetermined threshold.

9. The method of claim 1 wherein energy transferred to the tissue by the vapor causes coagulative necrosis of the nodule.

10. The method of claim 1 wherein delivering the vapor comprises delivering the vapor at a flow rate of between about 20 calories/second to about 200 calories/second.

11. The method of claim 1 wherein delivering the vapor comprises delivering the vapor for a duration of between about 2 seconds to about 5 minutes.

12. The method of claim 1 wherein delivering the vapor comprises delivering the vapor for a duration of between about 4 seconds to about 10 seconds.

13. The method of claim 1 further comprising generating the vapor in a generator disposed outside of the patient.

14. The method of claim 5 wherein the dose is between about 5 cal/g and about 20 cal/g.

15. The method of claim 5 wherein the dose is between about 5 cal/g and about 10 cal/g.

16. The method of claim 5 wherein the dose is between about 20 cal/g and about 40 cal/g.

17. The method of claim 1 wherein the inserting step comprises forming a channel into the lung region with the vapor delivery needle.

18. The method of claim 17 wherein the forming a channel step comprises forming a channel by puncturing through a pleural surface.

19. The method of claim 1 wherein the inserting step comprises puncturing the lung with the vapor delivery needle.

20. The method of claim 1 further comprising adjusting a vapor flow parameter during the delivering step to increase precision of vapor delivery.

21. The method of claim 20 wherein the adjusting step comprises adjusting a vapor quality.

22. The method of claim 20 wherein the adjusting step comprises adjusting a vapor momentum.

23. The method of claim 20 wherein the adjusting step comprises pulsing a vapor quality, a vapor flow rate, or a vapor momentum.

24. The method of claim 1 wherein the inserting step comprises inserting the vapor delivery needle directly into the diseased nodule.

25. The method of claim 1 wherein the delivering vapor step comprises delivering vapor through the vapor delivery needle directly into the diseased nodule to ablate the diseased nodule.

26. The method of claim 1 wherein the inserting step comprises positioning the needle near the diseased nodule, and wherein the delivering step comprises delivering vapor through a parenchyma of the lung to ablate the diseased nodule.

27. The method of claim 1 further comprising, prior to the inserting step, forming a channel in a parenchyma of the lung.

28. The method of claim 27 wherein the inserting step comprises positioning the needle near the diseased nodule, and wherein the delivering step comprises delivering vapor through the channel in the parenchyma to ablate the diseased nodule.

29. A method of treating disease of the lung, the method comprising:
imaging at least one nodule to be treated of the lung tissue;
determining a mass or a diameter of the nodule to be treated based on the imaging;
determining a safe and efficacious dosage of vapor for treating the nodule based on the mass or diameter of the nodule;
inserting a vapor delivery system into the lung proximate to the nodule to be treated; and
delivering the safe and efficacious dosage of vapor from the vapor delivery system to the nodule to be treated to ablate the nodule.

30. The method of claim 29 wherein the inserting step further comprises inserting a vapor delivery needle of the vapor delivery system into the lung proximate or within to the nodule to be treated.

31. A system for treating disease of the lung, the system comprising:
a vapor generator adapted to generate a heated water vapor;
a delivery catheter coupled to the vapor generator;
a vapor delivery needle disposed on a distal end of the delivery catheter, the vapor delivery needle configured to be inserted into a lung region; and
an electronic controller configured to determine a dosage of vapor to be delivered to a lung nodule based on a mass or diameter of the nodule, the controller also configured to determine a duration for delivering the vapor based on the dosage and an energy flow rate of the vapor generator.
